(19) United States
(12) Patent Application Publication
(10) Pub. No.: US 2026/0108647 A1
(43) Pub. Date: Apr. 23, 2026

(54) AUTOMATED APPARATUS FOR HANDLING RIGID STERILIZATION CONTAINERS IN DENTAL INSTRUMENTS STERILIZATION APPLICATIONS

(71) Applicant: Zeeno Robotics, LLC, Atlanta, GA (US)

(72) Inventors: David Ben-David, Rehovot (IL); Marc Lemchen, New York, NY (US)

(21) Appl. No.: 19/346,002

(22) Filed: Sep. 30, 2025

Related U.S. Application Data
(63) Continuation-in-part of application No. 17/671,078, filed on Feb. 14, 2022, now Pat. No. 12,447,223.
(60) Provisional application No. 63/151,155, filed on Feb. 19, 2021.

(51) Int. Cl. A61L 2/24; A61C 19/02; A61L 2/07; A61L 103/15; B08B 3/12; B08B 13/00; B25J 15/06

(52) U.S. Cl. CPC A61L 2/24; A61C 19/02; A61L 2/07; B08B 3/12; B08B 13/00; B25J 15/0608; B25J 15/0616; A61L 2103/15; A61L 2202/14; A61L 2202/17

(57) ABSTRACT

An automated system and method for efficiently cleaning, organizing, and sterilizing medical instruments including but not limited to surgical, dental, and orthodontic instruments. The sterilization center includes an automated apparatus for handling stacked rigid sterilization containers. The apparatus separates a container from a vertical stack, opens the container's latches and lid, enables instrument or cassette loading, and subsequently recloses and relatches the container while using a single-axis motion system. Actuation may be electric or pneumatic, and container gripping is achieved via suction, mechanical, or magnetic means. Stack management is achieved using horizontally actuated limiters. Latch actuation is accomplished via contact with stationary cams during the vertical movement.

This application claims priority to, and the benefit of the earlier filing date of US non-provisional application entitled “System and Method for Automated Sterilization of Dental and Orthodontic Instruments”, filed Feb. 14, 2022, Ser. No. 17/671,078, which in turn claims priority to, and the benefit of the earlier filing date of US provisional patent application entitled “System and Method for Automated Sterilization of Dental and Orthodontic Instruments”, filed on Feb. 19, 2021, Ser. No. 63/151,155, the contents of all of which are incorporated herein by reference.

### BACKGROUND

### Field of the Technology

The invention relates to the field of automated or robotic systems, specifically to automated dental sterilization systems, and more specifically, to an apparatus for handling, opening, loading, closing, and releasing rigid sterilization containers that are stacked one atop the other, using a single-axis motion system.

### Description of the Prior Art

Medical institutions such as hospitals, health clinics, doctors' offices, as well as dentist and orthodontist offices include a cleaning or sterilization center which is dedicated to cleaning and sterilizing the various medical or dental instruments used in their practice. After any procedure is performed on a patient, the medical instruments used must be thoroughly cleaned and then sanitized before they can be reused on another patient. What is specifically included within the sterilization center can vary according to the specific type of practice, however most commonly the sterilization center comprises a countertop, table, or work space comprising a number of different means for cleaning their instruments. These instrument cleaning means include but are not limited to ultrasonic cleaners, alcohol baths, washers, sinks, and/or autoclaves among others.

Previously, to use the sterilization center, a trained technician, nurse, or medical assistant must manually clean and organize each instrument individually by first removing any trash or debris from the trays containing the contaminated instruments and then pre-washing each instrument, either by manually wiping each instrument with alcohol or by placing the instrument in an alcohol bath or ultrasonic cleaner. In some instances, such as in dentist and orthodontist offices, after being pre-washed the technician must then orientate and place each instrument in a corresponding space within a cassette. The cassette is then placed in a washer specially designed for such a purpose. Next, after the cleaning cycle is complete, the technician must remove the cassettes from the washer and drop them into a sterilization bag which is then placed inside an autoclave or other machine or device which is capable of sterilization through application of high pressure and temperatures. After the autoclave has completed its sterilization cycle, the technician removes the bagged cassettes from the autoclave and then places them on a drying or cooling rack or beneath other drying/cooling means such as a fan.

Traditional sterilization methods involve wrapping cassettes or instruments in medical-grade sterilization paper or sealing them in disposable pouches prior to autoclaving. While common, these manual processes are labor-intensive and pose several limitations and potential hazards to the staff. Specifically, the wrapping process is time-consuming, error-prone, and often leads to inconsistent sealing or tearing, compromising sterility. Moreover, single-use packaging materials contribute to substantial medical waste and recurring costs.

Rigid sterilization containers are an FDA and CDA approved alternative. These containers are durable, reusable, and provide a consistent sterile barrier for instruments. Designed with mechanical latches and removable lids, rigid containers eliminate the need for wrap or pouch materials, improve workflow repeatability, and reduce both environmental footprint and long-term cost.

Despite their advantages, the use of rigid containers in automated or robotic dental instrument sterilization systems is unexplored, requiring complex movements for opening, closing, loading and unloading the containers. Additionally, existing automated sterilization systems do not address the unique mechanical challenges of rigid container manipulation, particularly in stacked arrangements.

As can be seen from the above, the process of cleaning and sterilizing is time consuming and laborious. In addition, it requires a trained technician whose time and skill can be used more effectively elsewhere to focus solely on the monotonous yet essential task of sterilizing instruments. The burden of cleaning and sterilization is further magnified when the instrument is too large or bulky to fit within a cassette, thereby requiring the technician to wash the instrument by hand and/or run a sterilization cycle with only that particular instrument disposed in the autoclave, both of which in turn further decreases the efficiency of the technician's ability to sterilize instruments. Additionally, the sterilization of medical instruments can also present an occupational hazard to the technician can accidentally puncture or cut their hands or fingers with medical instruments which may not be fully cleaned or sterilized. Because of the inherent risk associated with unsterilized instruments, the technician must further record or document the sterilization process for each instrument for legal liability purposes, thereby further increasing the time needed to properly clean a set of medical instruments.

What is needed therefore is an automated system and method which effectively and efficiently cleans, organizes, and sterilizes medical instruments with only minimal support and oversight from a technician or other human operator. The system and method should be able to perform each of the tasks previously performed by the technician and should also be able to be adaptable so as to clean larger or more bulky instruments in addition to more commonly used instruments. Additionally, the system and method should provide a means for a user to track and monitor any specific instrument or group of instruments as they progress through the cleaning and sterilization process and to further document its successful completion.

### BRIEF SUMMARY

The current invention provides an automated system for cleaning or sterilizing at least one instrument. The system includes a plurality of cleaning or sterilization devices that are each configured to clean or sterilize instruments that are disposed within a work area and at least one robotic element disposed within the work area. The robotic element itself is configured to traverse a length within the work area, transport the instruments between at least two of the plurality of cleaning or sterilization devices, and then operate at least one of the cleaning or sterilization devices.

In certain embodiments, an apparatus is provided that automates the handling of stacked rigid sterilization containers for use in dental or medical instrument reprocessing systems. The invention enables fully automated separation, opening, loading, closing, and locking of the containers using a single-axis actuator system, thereby streamlining sterilization workflows and allowing integration with automated and robotic sterilization setups.

In certain embodiments, the apparatus is capable of separating a single container from a vertically stacked set, opening the container's mechanical latches using a passive cam mechanism, lifting the container lid to create access for instrument loading, allowing instruments or cassettes to be inserted by a robotic arm, gantry system, or automated loader, and reclosing and relatching the container in a fully automated sequence.

The entire process may be actuated via a single vertical motion axis, which may be driven by a stepper motor, servo motor, DC motor, or pneumatic or hydraulic cylinder. Containers may be gripped using vacuum suction cups, electromagnetic grippers, mechanical clamps, or a combination thereof.

In certain embodiments, stack management may be accomplished using a pair of horizontal limiters or latches at the base of the container stack that may be engaged and disengaged by linear motion.

According to certain embodiments, the apparatus dramatically improves the automation potential of sterilization rooms and central sterilization departments by enabling seamless integration with robotic workflows, reducing risk of injury, lowering waste, and improving consistency and efficiency of instrument handling.

In certain embodiments, an automated system for cleaning or sterilizing at least one instrument is provided. The system includes a plurality of cleaning or sterilization devices configured to clean or sterilize instruments disposed within a work area, an automated apparatus disposed within the work area that is configured to receive a plurality of containers in a stacked configuration, where the containers are each in turn configured to receive at least one instrument, and at least one robotic element disposed within the work area. The at least one robotic element is configured to traverse a length within the work area, insert or remove the containers to or from the automated apparatus, transport the at least one instrument between at least two of the plurality of cleaning or sterilization devices, and operate at least one of the plurality of cleaning or sterilization devices.

In certain embodiments, a method for engaging a container for use within an automated system for cleaning or sterilizing medical instruments is provided. The method includes providing a plurality of containers in a stacked configuration, selectively gripping a first container from the plurality of containers in the stacked configuration, lowering the first container relative to the remaining plurality of containers in the stacked configuration, and engaging a lid portion of the first container with a pair of engagement portions. The method may also include separating the lid portion from a body portion of the first container and accessing an interior of the body portion by at least one robotic element of the automated system.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a front view of a first embodiment of the current invention comprising a plurality of robotic arms disposed on a frame which is in turn mounted or disposed above a pre-existing cleaning and sanitization center within a dental or medical office.

FIG. 2 is a top down view of the current invention seen in FIG. 1, according to an embodiment.

FIG. 3 is a flow diagram illustrating a method of use of the current invention when cleaning and sterilizing one or more dental or medical instruments, according to an embodiment.

FIG. 4 is a flow diagram illustrating an alternative embodiment related to the use of the current invention when cleaning and sterilizing one or more specialized or large dental or medical instruments.

FIG. 5 is a perspective view of a container stack disposed within a rigid container sterilization apparatus that is part of the sanitization center, according to an embodiment.

FIG. 6 is a cross-sectional side view of the container stack and the rigid container sterilization apparatus seen in FIG. 5 with limiters of the apparatus engaged, according to an embodiment.

FIG. 7 is a cross-sectional side view of the container stack and the rigid container sterilization apparatus with a motion axis of the apparatus engaging and lowering the bottom container as well as container re-engagement with lid and latch closure cams, according to an embodiment.

FIG. 8 is a cross-sectional side view of the container stack and the rigid container sterilization apparatus with a cam mechanism of the apparatus interacting with container latches, according to an embodiment.

FIG. 9 is a cross-sectional side view of the container stack and the rigid container sterilization apparatus illustrating lid separation via a cam and motion axis of the apparatus, according to an embodiment.

FIG. 10 is a perspective view of the container stack and the rigid container sterilization apparatus illustrating an instrument loading phase, according to an embodiment.

FIG. 11 is a perspective view of a rigid sterilization container with an open lid, according to an embodiment.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

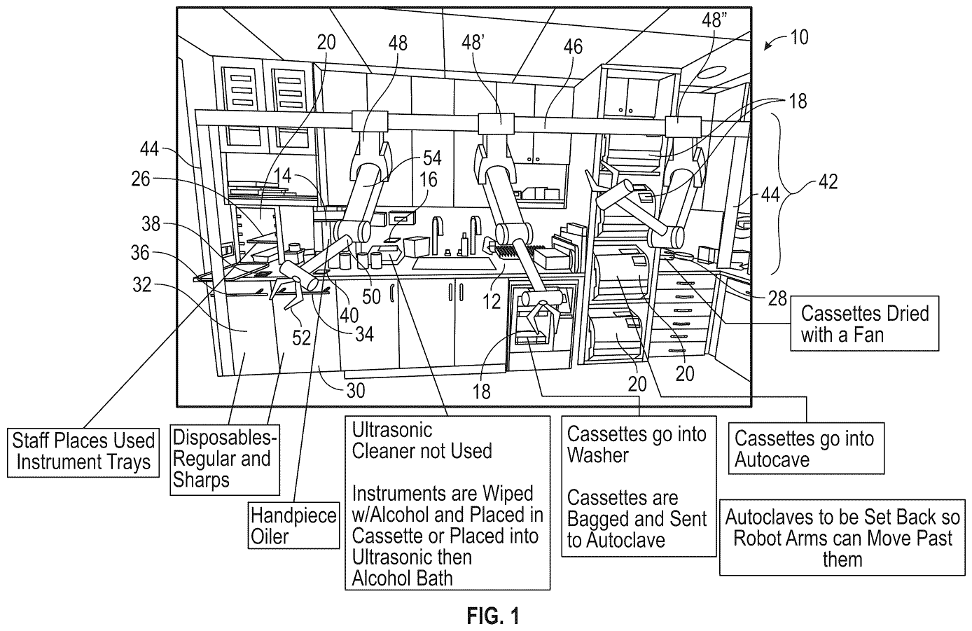

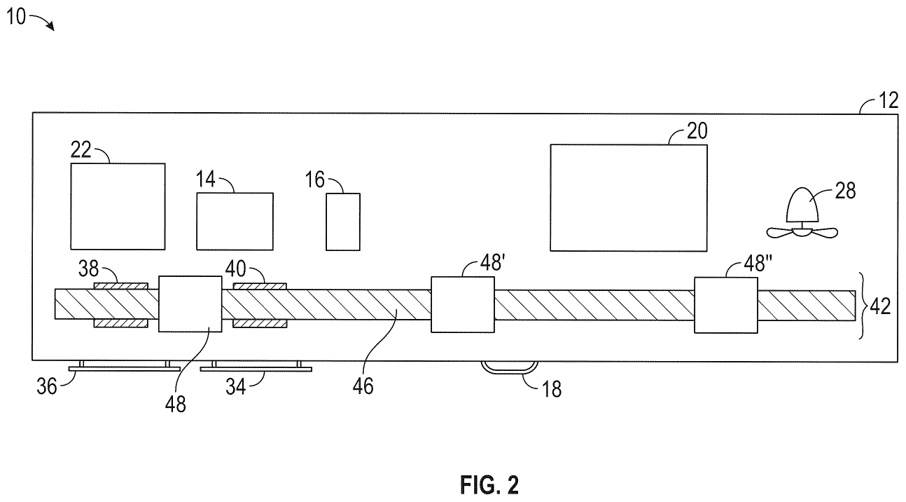

The current invention is an automated system and method for efficiently cleaning, organizing, and sterilizing medical instruments including but not limited to surgical, dental, and orthodontic instruments. Greater detail of the system may be had by turning to FIGS. 1 and 2 which show a frontal view and a top-down view of a sterilization center 10, respectively. The sterilization center 10 comprises a counter top or work top 12 on which is disposed a plurality of well-known devices for cleaning, maintaining, and sterilizing instruments including but not limited to a handpiece oiler 14, an ultrasonic cleaner 16, an instrument washer 18, and at least one autoclave 20 or other sterilization device. Also disposed on the work top 12 is a rack 22 which is capable of storing a plurality of trays 24 in a stacked configuration, each of the plurality of trays 24 containing one or more instruments 26. Disposed on an opposing end of the work top 12 is fan 28 which is used to dry off and cool down the instruments 26 once they have been removed from one of the autoclaves 20. As shown in FIG. 1, disposed beneath or under the work top 12 is a plurality of additional devices or containers, including but not limited to a first waste container 30 for general refuse and a separate, second waste container 32 for sharp items or hazardous materials. The first and second waste containers 30, 32 may be accessed by pulling their respective handles 34, 36 and then depositing the refuse, or alternatively, by dropping the refuse through a corresponding gap or hole 38, 40 defined in the upper surface of the work top 12.

It should be noted that the type, number, and exact placement of the various cleaning and sterilization devices within the sterilization center 10 as seen in FIGS. 1 and 2 is meant to be illustrative purposes only and that the specific configuration of the sterilization center 10 may be specifically tailored for each health professional's specific type of practice or needs. For example, one health professional may require a sterilization center 10 with at least four autoclaves 20 disposed in a stacked configuration as seen in FIG. 1, however another health professional may only require two and may not have the space for a stacked configuration and so instead must dispose them side by side. In another example, one type of health professional may not need an ultrasonic cleaner 16 at all and instead may require another type of cleaning device which is not explicitly disclosed herein but which may be incorporated into the sterilization center 10 in its place. It is therefore to be expressly understood that different configurations of the sterilization center 10 other than what is explicitly shown are well within the spirit and scope of the current invention.

Returning to FIGS. 1 and 2, the sterilization center 10 further comprises a frame 42 coupled to or embedded in the work top 12. The frame 42 itself comprises a plurality of vertical posts 44 and at least one horizontal traverse or track 46. Disposed or coupled to the horizontal traverse 46 are a plurality of robotic arms or elements 48, 48′, 48″. Each robotic arm or element 48, 48′, 48″ preferably comprises an upper portion 54 and a lower portion 50 with a hand or other gripping tool 52 coupled to a distal end of the lower portion 50. Each robotic arm or element 48 is further configured to traverse the horizontal track 46 in either lateral direction, thereby allowing each robotic arm or element 48, 48′, 48″ to be suspended above the work top 12 while moving across or over at least a portion of the length of the work top 12. Additionally, each robotic arm or element 48, 48′, 48″ may pivot, bend, or rotate between each joint disposed on either end of the upper portion 54 and the lower portion 50 accordingly so that the upper portion 54 and the lower portion 50 may access or reach each portion of the work top 12. The gripping tool 52 may be actuated to open, close, grip, or otherwise grab and hold an object between its calipers, pincers, or robotic fingers.

As seen in FIGS. 1 and 2, the frame 42 comprises two vertical posts 44 and single horizontal traverse or track 46, however it is to be expressly understood that fewer or additional posts 44 or tracks 46 may be used in order to best suit the particular sterilization center 10 the current invention is installed or placed in. Similarly, while three robotic arms or elements 48, 48′, 48″ are seen in FIGS. 1 and 2, fewer or additional robotic arms or elements 48 may be disposed along the track 46 so as to provide the most efficient means possible for sterilizing the health professional's instruments 26.

The plurality of robotic arms or elements 48, 48′, 48″ are controlled and monitored by an external controller such as a computer, smart device, or other means which is communicated to the robotic arms or elements 48 through a wireless connection such as Bluetooth®. The external controller contains the instructions for each robotic arm or element 48, 48′, 48″ to clean, organize, and sterilize the instruments 26 and allows the health professional to begin or end the sterilization process when needed. Additionally, the external controller further comprises an input means for the health professional to edit, amend, or change the specific operational instructions or programming for one or all of the robotic arms or elements 48, 48′, 48″.

It should be noted that the robotic arms or elements 48, 48′, 48″ and its corresponding frame 42 are meant to be for illustrative purposes only and that fewer, additional, or different robotic elements may used in conjunction with or instead of said elements. For example, instead of the robotic arms 48, 48′, 48″, the robotic elements may instead be a plurality of wheeled or aerial robots as disclosed in U.S. Provisional Application 63/291,764 entitled “System and Method for Robotic Office Management” which is herein incorporated by reference in its entirety. Such a system or network of a plurality of robots comprising trays, means for actuation, and navigation means could be used to work in tandem to complete a series of tasks related to the cleaning and sanitizing of medical instruments that is controlled from a single control point or terminal.

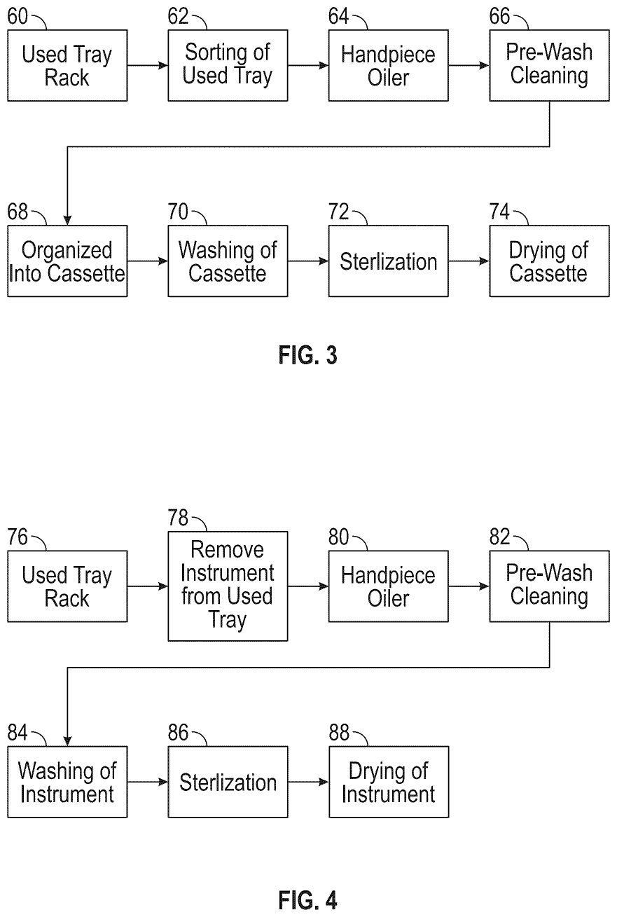

Further understanding of how the current invention operates may be seen from the flow diagrams presented in FIGS. 3 and 4. Starting with FIG. 3, the health professional or other means such as a mobile robot or automated machine begins the current sterilization method by first placing the used trays 24 containing one or more contaminated instruments 26 into the rack 22 in step 60. After initializing the sterilization process at the external controller, a first robotic arm or element 48 traverses across the track 46 until it is sufficiently disposed in front of the rack 22 where it then removes the tray 24 containing the contaminated instruments 26. Then, with either the assistance of a second robotic arm or element 48′ or by placing the tray 24 onto the work top 12, the first robotic arm or element 48 sorts through the contents of the tray 24 in step 62. In this step, general refuse is picked up by the gripping tool 52 of the first robotic arm or element 48 and placed or dropped into the first waste container 30 while other hazardous materials such as used syringes and the like are picked up by the gripping tool 52 and then placed or dropped into the second waste container 32. After sorting the contents of the tray 24, the only items left in the tray are the contaminated instruments 26 which are to be cleaned and sterilized.

Next, in step 64, the robotic arm or element 48 which comprises an RFID reader, barcode scanner, or other recognition means such as image instrument recognition, scans or reads an RFID tag or barcode disposed on or within each of the instruments 26 in order to identify specific instruments, such as for example dental handpieces, which require lubrication. In one particular embodiment, a camera disposed on the robotic arm or element 48 may be used in conjunction with an AI image recognition program so as to recognize or identify a specific instrument 26 by its unique shape. The robotic arm or element 48 then removes those particular instruments 26 from the tray 24 and places them into the handpiece oiler 14. In one particular embodiment, the robotic arm or element 48 is programmed or trained to operate or set the handpiece oiler 14. Alternatively, the handpiece oiler 14 may be specifically designed or configured to operate in conjunction with the robotic arm or element 48 so that once the instruments 26 are inserted into the handpiece oiler 14, the handpiece oiler 14 automatically begins the lubrication process. Once the lubrication process has been completed, the robotic arm or element 48 removes the instruments 26 from the handpiece oiler 14 and returns them to the tray 24 or to another portion of the work top 12.

In step 66, the robotic arm performs a preliminary wash of the instruments 26. In one preferred embodiment, the robotic arm wipes each instrument 26 with alcohol thereby removing any relatively large debris disposed thereon. In a related embodiment, each instrument 26 is instead or additionally placed into an alcohol bath disposed on the work top 12. In yet another embodiment, instead of wiping each instrument 26 with alcohol, the robotic arm or element 48 may first place one or more of the instruments 26 into the ultrasonic cleaner 16 and then drop the instrument into an alcohol bath once the ultrasonic cleaning process has been completed.

Following step 66, the robotic arm 48 begins to organize the now pre-cleaned instruments 26 and prepare them for further washing and sterilization in step 68. The robotic arm or element 48, using the equipped RFID reader, barcode scanner, or other scanning means, reads each the RFID tag or barcode disposed on each instrument 26 and matches or associates each instrument with a predetermined cassette and/or a predetermined position within a cassette. Specifically, after initially scanning the RFID tag on a specific instrument 26, the resulting reading is matched against a database or set of instructions contained either within the robotic arm or element 48 or within the external controller which dictates not only which specific cassette the instrument 26 is to be placed in, but also which predetermined position and orientation within that cassette the instrument 26 is to be specifically inserted. The robotic arm or element 48 then scans or reads a corresponding RFID tag or barcode disposed on the cassette to ensure that it is the correct cassette. If the cassette does not match the corresponding ID for the cassette meant for the previously scanned instrument 26, the cassette is placed to the side or removed from the work top 12 and a new cassette is scanned to determine if it is the proper cassette for the currently selected instrument 26. If the cassette does match the corresponding ID for the cassette meant for the previously scanned instrument 26, the robotic arm 48 opens the cassette and then places the scanned instrument 26 into the cassette accordingly. A plurality of secondary RFID tags or barcodes may be distributed throughout the inside of each cassette so that after the robotic arm or element 48 has placed and/or secured the instrument 26 to an internal portion of the cassette, an adjacent secondary RFID tag or barcode is scanned to ensure that the instrument 26 has been placed at the correct pre-determined location and orientation within that particular cassette. If there is a mismatch between the scanned secondary RFID tag or barcode and the specific ID contained within the external controller, the robotic arm or element 48 removes the instrument 26 and rescans the remaining RFID tags or barcodes within the cassette to determine where the correct position is. This automated organizing process is repeated until the prerequisite number or type of instruments 26 have been correctly inserted into the cassette. The specific number or type of instruments 26 which are to be placed within the cassette may be determined by a predetermined threshold amount, however in a preferred embodiment, each RFID tag or barcode corresponding to the cassette contains a list or predetermined set of instruments 26 that are for a specific related treatment or procedure. For example, one cassette may be dedicated to housing or accommodating instruments 26 related or needed for a dental cleaning, while another cassette may be dedicated to housing or accommodating instruments 26 related to orthodontic bracket removal.

Next in step 70, each cassette which is now full of pre-washed instruments 26, is closed and then placed by the robotic arm or element 48 into the instrument washer 18. In one embodiment, the first robotic arm or element 48 traverses across the length of the work top 12 via the suspended horizontal track 46 until it is disposed in front of the instrument washer 18 where the robotic arm or element 48 can then insert the cassette therein. Alternatively, after the organization and placement of the instruments in step 68 has been completed, the second robotic arm or element 48′ may instead pick up the cassette using its corresponding gripping tool 52, traverse the length of the work top 12 via the suspended horizontal track, and then insert the cassette into the instrument washer 18, thereby leaving the first robotic arm or element 48 free to return to the rack 22 and repeat steps 60-68 with a new batch of instruments 26. In yet another embodiment, the first robotic arm or element 48 and the second robotic arm or element 48′ may work in conjunction with one another in order to complete step 70. For example, after completing step 68, the first robotic arm or element 48 or the external controller signals or prompts the second robotic arm or element 48′ which then moves over to the instrument washer 18 and opens it, allowing the first robotic arm or element 48 to approach and then insert the cassette therein. The instrument washer 18 is then initiated by either the first or second robotic arm or element 48, 48′, or alternatively, the instrument washer 18 may be configured to automatically begin its washing cycle immediately after being closed by either of the robotic arms or elements 48, 48′.

Once the instrument washer 18 has completed its wash cycle, the second robotic arm or element 48′ in step 72 as seen in FIG. 3 removes the cassette from the instrument washer 18 and then places it in a sterilization bag. The bagged cassette is then dropped or placed by the second robotic arm or element 48′ into one of the plurality of autoclaves 20 which is then initiated and which performs a high-pressure sterilization of the bagged cassette. In a related embodiment, instead of the second robotic arm or element 48′ removing each cassette from the instrument washer 18 and placing it into an autoclave 20, a third robotic arm or element 48″ may be used to perform the same task which in turn allows the second robotic arm or element 48′ to perform a different task or to return to the pre-wash portion of the work top 12 and place a new, different cassette into the instrument washer 18.

In step 74, after the autoclave 20 has completed its sterilization cycle, the second or third robotic arm or element 48′, 48″ removes the bagged cassette from the autoclave 20. Next, the bagged cassette is then placed into a dedicated drying area or rack where the fan 28 may finish the drying/cooling process of the instruments 26. Alternatively, any of the robotic arms or elements 48, 48′, 48″ may simply hold the bagged cassette after it has been removed from the autoclave 20 until it is sufficiently cool for a health professional or mobile robot to remove it from the sterilization center 10. After the drying process is complete, the third robotic arm or element 48″ may place the bagged cassette into a storage area, however in preferred embodiment, the third robotic arm or element 48″ picks up the now dried bagged cassette and places it into a rack or onto a cart where it may be stored until needed or until the health professional removes it from the sterilization center 10. In a further embodiment, the third robotic arm or element 48″ may scan the RFID tag or barcode disposed on the cassette which notifies the external controller and/or the health professional that the scanned bagged cassette has completed the sterilization process and is now ready for immediate use. In a particular embodiment, the system tracks and monitors the number of cassettes and/or the number of instruments 26 which are available for immediate patient use. The system may further be integrated with a practice management program to ensure that the correct type and number of instruments 26 needed for any upcoming patient procedures are cleaned, sterilized, and otherwise made available before the scheduled procedure.

In a related embodiment depicted in FIG. 4, the current system and method may be used to directly clean and sterilize specialized instruments 26′ which are too large or bulky to fit within a cassette, for example cheek retractors, bur blocks, and the like.

In this modified method, the health professional or mobile robot begins the process by first placing the used trays 24 containing one or more contaminated specialized instruments 26′ into the rack 22 or another type of container such as a bin in step 76. After initializing the sterilization process at the external controller, a first robotic arm or element 48 traverses across the track 46 until it is sufficiently disposed in front of the rack 22 where it then removes the tray 24 containing the contaminated specialized instruments 26′. Then, with either the assistance of a second robotic arm or element 48′ or by placing the tray 24 onto the work top 12, the first robotic arm or element 48 sorts through the contents of the tray 24 in step 78. In this step, general refuse is picked up by the gripping tool 52 of the first robotic arm or element 48 and placed or dropped into the first waste container 30 while other hazardous materials such as used syringes and the like are picked up by the gripping tool 52 and then placed or dropped into the second waste container 32. After sorting the contents of the tray 24, the only items left in the tray are the contaminated specialized instruments 26′ to be cleaned and sterilized.

Next, in step 80, the robotic arm or element 48 which comprises an RFID reader, barcode scanner, or other recognition means, scans or reads an RFID tag or barcode disposed on each of the specialized instruments 26′ in order to identify specific instruments such as dental handpieces which require lubrication. The robotic arm or element 48 then removes those particular specialized instruments 26′ from the tray 24 and places them into the handpiece oiler 14. In one particular embodiment, the robotic arm or element 48 is programmed or trained to operate or set the handpiece oiler 14. Alternatively, the handpiece oiler 14 may be specifically designed or configured to operate in conjunction with the robotic arm or element 48 so that once the specialized instruments 26′ are inserted into the handpiece oiler 14, the handpiece oiler 14 automatically begins the lubrication process. Once the lubrication process has been completed, the robotic arm or element 48 removes the specialized instruments 26′ from the handpiece oiler 14 and returns them to the tray 24 or other portion of the work top 12.

In step 82, the robotic arm performs a preliminary wash of the specialized instruments 26′. In one preferred embodiment, the robotic arm or element 48 wipes each specialized instrument 26′ with alcohol thereby removing any relatively large debris disposed thereon. In a related embodiment, each specialized instrument 26′ is instead or additionally placed into an alcohol bath disposed on the work top 12. In yet another embodiment, instead of wiping each specialized instrument 26′ with alcohol, the robotic arm or element 48 may first place one or more of the specialized instruments 26′ into the ultrasonic cleaner 16 and then drop the instrument into an alcohol bath once the ultrasonic cleaning process has been completed.

Next in step 84, each specialized instrument 26′ which is now fully pre-washed, is placed by the robotic arm or element 48 into the instrument washer 18. In one embodiment, the first robotic arm or element 48 traverses across the length of the work top 12 via the suspended horizontal track 46 until it is disposed in front of the instrument washer 18 where the robotic arm or element 48 can then insert the specialized instrument 26′. Alternatively, the second robotic arm or element 48′ may instead pick up the specialized instrument 26′ using its corresponding gripping tool 52, traverse the length of the work top 12 via the suspended horizontal track, and then insert the specialized instrument 26′ into the instrument washer 18, thereby leaving the first robotic arm or element 48 free to return to the rack 22 and repeat steps 76-82 with a new specialized instrument 26′. In yet another embodiment, the first robotic arm 48 and the second robotic arm or element 48′ may work in conjunction with one another in order to complete step 84. For example, after completing step 82, the first robotic arm or element 48 or the external controller signals or prompts the second robotic arm or element 48′ which then moves over to the instrument washer 18 and opens it, allowing the first robotic arm or element 48 to approach and then insert the specialized instrument 26′ therein. The instrument washer 18 is then initiated by either the first or second robotic arm or element 48, 48′, or alternatively, the instrument washer 18 may be configured to automatically begin its washing cycle immediately after being closed by either of the robotic arms or elements 48, 48′.

Once the instrument washer 18 has completed its wash cycle, the second robotic arm or element 48′ in step 86 as seen in FIG. 4 removes the specialized instrument 26′ from the instrument washer 18 and then places it into a sterilization bag. The bagged specialized instrument 26′ is then dropped or placed by the second robotic arm or element 48′ into one of the plurality of autoclaves 20 which is then initiated and which performs a high-pressure sterilization of the bagged specialized instrument 26′. In a related embodiment, instead of the second robotic arm or element 48′ removing each specialized instrument 26′ from the instrument washer 18 and placing it into an autoclave 20, a third robotic arm or element 48″ may be used to perform the same task which in turn allows the second robotic arm or element 48′ to perform a different task or to return to the pre-wash portion of the work top 12 and place a new, different specialized instrument 26′ into the instrument washer 18.

In step 88, after the autoclave 20 has completed its sterilization cycle, the second or third robotic arm or element 48′, 48″ removes the bagged specialized instrument 26′ from the autoclave 20. Next, the bagged specialized instrument 26′ is then placed into a dedicated drying area where the fan 28 may finish the drying process and further cool the bagged specialized instruments 26′. Alternatively, any of the robotic arms or elements 48, 48′, 48″ may simply hold the bagged specialized instrument 26′ after it has been removed from the autoclave 20 until it is sufficiently cool for a health professional to remove it from the sterilization center 10. After the drying process is complete, the third robotic arm or element 48″ may remove the bagged specialized instruments 26′ and place them into a storage area, however in preferred embodiment, the third robotic arm or element 48″ picks up the now dried and bagged specialized instrument 26′ and places it into a rack or onto a cart where it may be stored until needed or until the health professional removes it from the sterilization center 10. In a further embodiment, the third robotic arm or element 48″ may scan the RFID tag or barcode disposed on or in the bagged specialized instrument 26′ which notifies the external controller and/or the health professional that the scanned specialized instrument 26′ has completed the sterilization process and is now ready for immediate use.

In a further related embodiment, the above discussed system and methods of operation are customizable and are configured to incorporate additional or new cleaning or sterilization devices as they come to market or otherwise gain widespread use. For example, a combined ultrasonic cleaner and washer may be incorporated into the sterilization center 10 which would effectively combine steps 66 and 70, thereby simplifying the method and perhaps even negating the need for having a second or third robotic arm or element 48′, 48″ within the system. Similarly, if the standards of practice were to change, the current system and method could likewise be adapted or altered to fit the current standard of care. For example, if there were no longer any regulations requiring that instruments be bagged before being sterilized, a washer combined with an autoclave could be introduced into the current system and method which would wash rinse and autoclave all in one step. The robotic arms or elements 48, 48′, 48″ could likewise be adapted or reprogrammed to operate a single washer/autoclave device and therefore eliminate any steps for moving the cassettes and/or instruments back and forth therebetween or for bagging the cassettes and/or instruments.

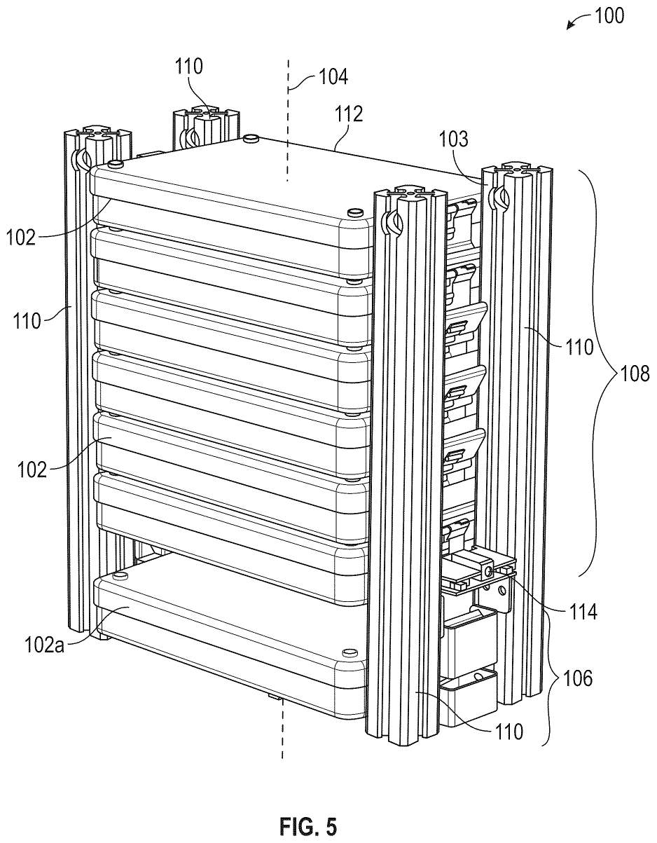

According to certain embodiments, the sterilization center 10 includes an automated apparatus 100 seen in FIG. 5 that is designed to handle stacked rigid sterilization containers 102 in an automated dental or medical environment. The apparatus 100 may include a motion axis 116 (not seen in FIG. 5) that may be vertically driven along or parallel to a vertical axis 104, the motion axis 116 being capable of performing all container manipulation steps using this motion axis 116.

In certain embodiments, containers 102 may be vertically stacked and supported above two side-mounted horizontal engagement portions 106 at the base or bottom portion of a stack 108. Disposed vertically above each of the engagement portions 106 may be a corresponding limiter 114 that may be configured to selectively hold or maintain the stack 108 within the automated apparatus 100. Each of the engagement portions 106 may engage the edges of a bottom or first container 102a while the limiter 114 may hold the weight of the stack 108 above. The stack 108 may include any number of containers 102 and is not limited to the number of containers 102 explicitly shown in the figures. Any lateral movement of the containers 102 which form the stack 108 may be prevented by a plurality of vertical posts 110 which form a type of support structure or frame 103 for the stack 108. The posts 110 may provide an aperture 112 which allow additional containers 102 to loaded into the automated apparatus 100 as needed.

According to certain embodiments, the engagement portions 106 may be actuated via a linear motion along the vertical axis 104, for example the motion axis 116 which may be powered by pneumatic cylinders, solenoids, electric motors, stepper motors, or servo motors.

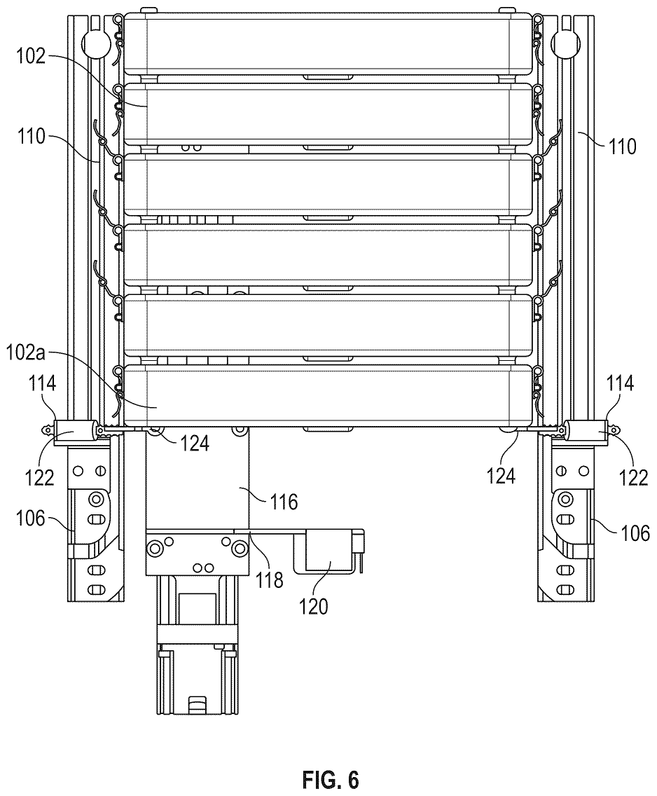

In certain embodiments, as seen in FIG. 6, the motion axis 116 may include a horizontally disposed platform 118 and a gripper 120 disposed on a end portion of the platform 118. When a lower most or first container 102a needs to be accessed, the gripper 120 mounted or coupled to the motion axis 116 grips the bottom of first container 102a. In certain embodiments, the gripper 120 may be an electromagnet as seen in FIGS. 6-9, however in other embodiments the gripper 120 may include vacuum actuated suction cups, other electromagnetic actuators, or other mechanical grippers.

For example, the process may begin with the motion axis 116 moving upward until the platform 118 is in contact with a bottom surface of the first container 102a. The gripper 120 then selectively acquires and maintains a hold or grip on the first container 102a. Once the first container 102a is securely held, the limiters 114 may be disengaged, allowing the entire stack 108 to rest on the gripped first container 102a. In certain embodiments, each limiter 114 may include a linear actuator 122 such as a solenoid, stepper motor, or electric motor, and a retention rod 124, the linear actuator 122 being configured to actuate or selectively retract and extend the retention rod 124. For example, when the limiters 114 are disengaged, the linear actuator 122 may be activated so as to the retract the retention rod 124, thereby allowing the first container 102a to move vertically downward relative to the stack 108.

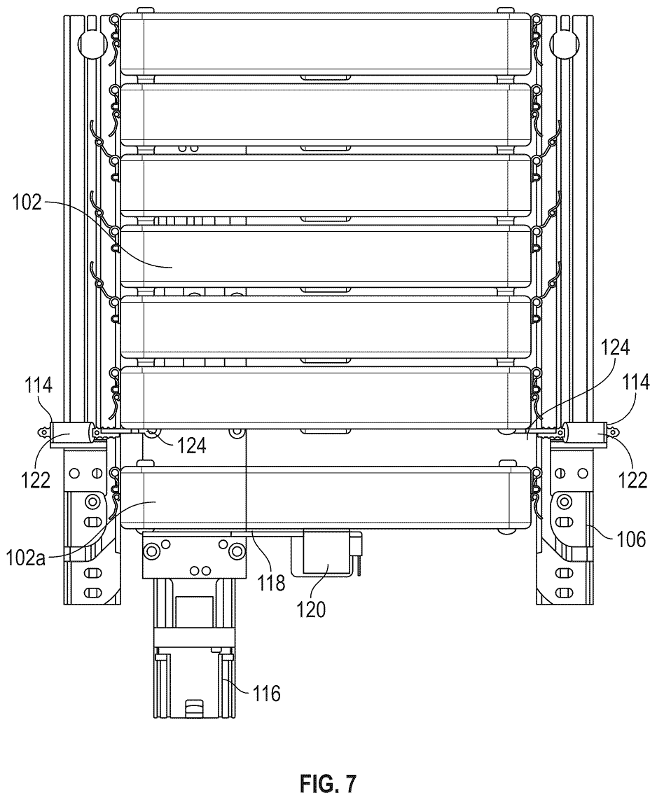

As seen in FIG. 7, the motion axis 116 then moves downward within the automated apparatus 100 by the height of one container 102, and the limiters 114 are re-engaged, thereby isolating or separating the rest of the stack 108 from the first container 102a. In certain embodiments, the gripper 120 may include a sensor or multiple sensors, such as a proximity sensor, an optical sensor, or a plurality of ultrasonic sensors configured to indicate when the first container 102a has been engaged or gripped successfully and securely by the gripper 120. In certain embodiments, the sensor, the motion axis 116, and/or the gripper 120 may be configured to transmit a digital or analog signal to the external controller of the sterilization center 10, for example.

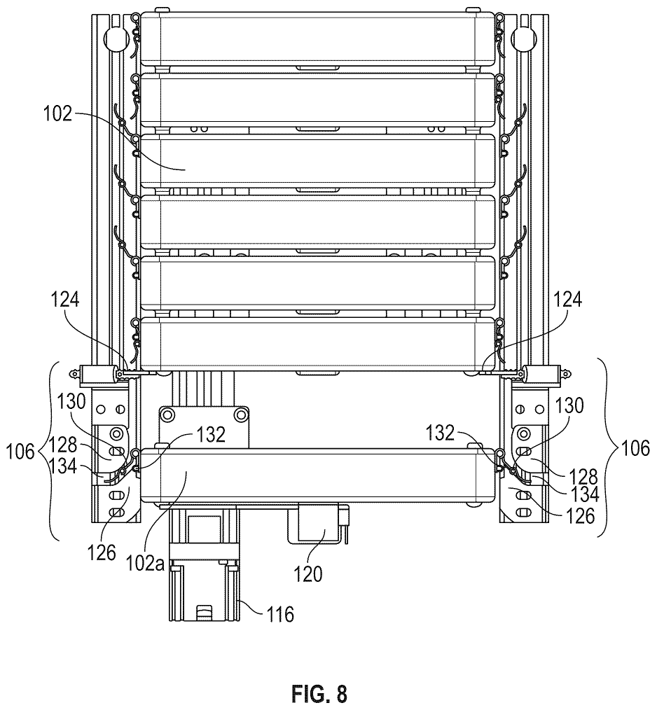

As seen in FIG. 8, as the motion axis 116 continues vertically downward with the first container 102a, contact may be made with the engagement portions 106 disposed on either lateral side of the automated apparatus 100. In certain embodiments, each engagement portion 106 includes two stationary cam profiles mounted along the sides of the automated apparatus 100, for example an opening cam 126 and a closing cam 128 with a channel 134 defined there between. Both the opening cam 126 and the closing cam 128 may be configured to engage a pair of corresponding mechanical latches 130 disposed on the first container 102a. In certain embodiments, the opening cams 126 may be shaped such that contact points along a surface 132 of the opening cams 126 induce a rotational force vector on the respective latches 130, causing them to rotate and open as the first container 102a is moved vertically downward via the motion axis 116 as seen in FIG. 8. For example, each of the latches 130 may initially be in a closed configuration where the latches 130 are directed downward while the first container 102a is in the stack 108. As the first container 102a is lowered, a tip portion of each latch 130 makes contact with the angled surface 132 of the opening cam 126.

Continued downward movement causes each latch 130 to rotate about its respective hinge within the channel 134 while the first container 102a continues to move downward past the opening cam 126. The opening and closing cams 126, 128 may include or be fabricated with stainless steel, coated carbon steel, tool steel, aluminum, high-performance polymers or plastics, or a combination thereof.

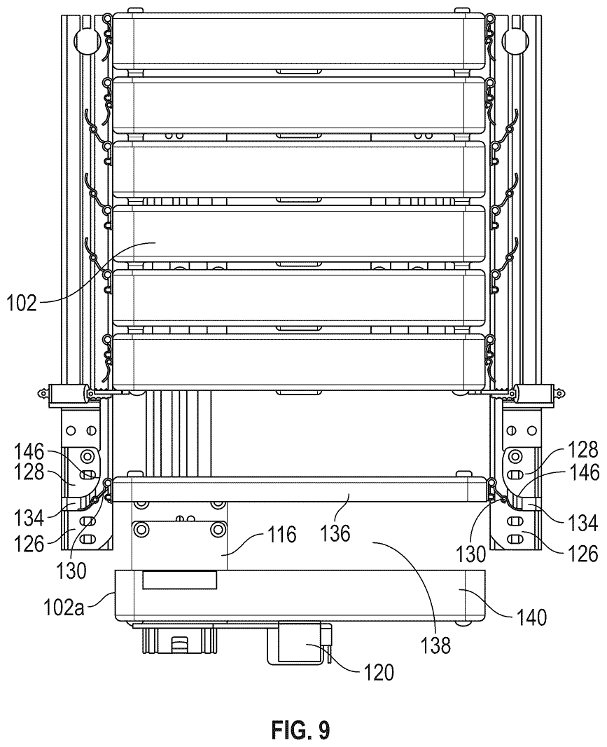

As illustrated in FIG. 9, once the latches 130 are fully opened, namely when the opening cam 126 has fully rotated each latch 130 to a maximum position within the channel 134, the geometry of the surface 132 of the opening cam 126 may be configured to support a lid 136 of the first container 102a in an open position. As the first container 102a continues downward, a gap 138 may be created between the lid 136 and a body 140, thereby exposing an interior 142 of the first container 102a as seen in FIG. 11.

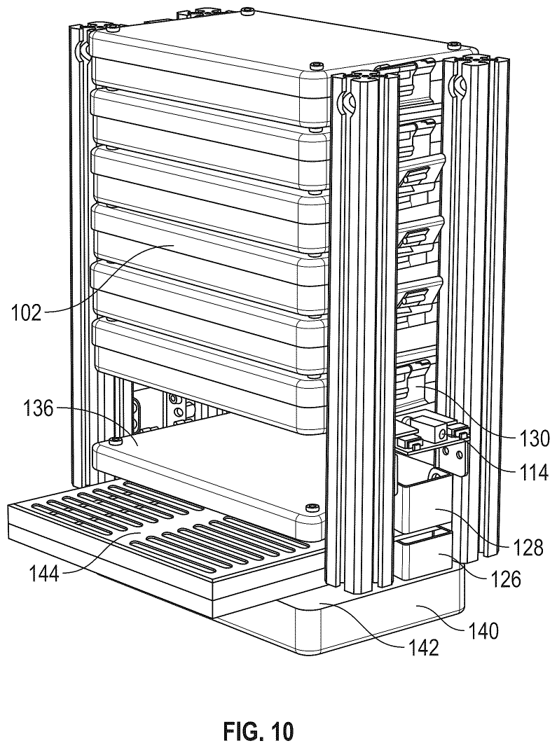

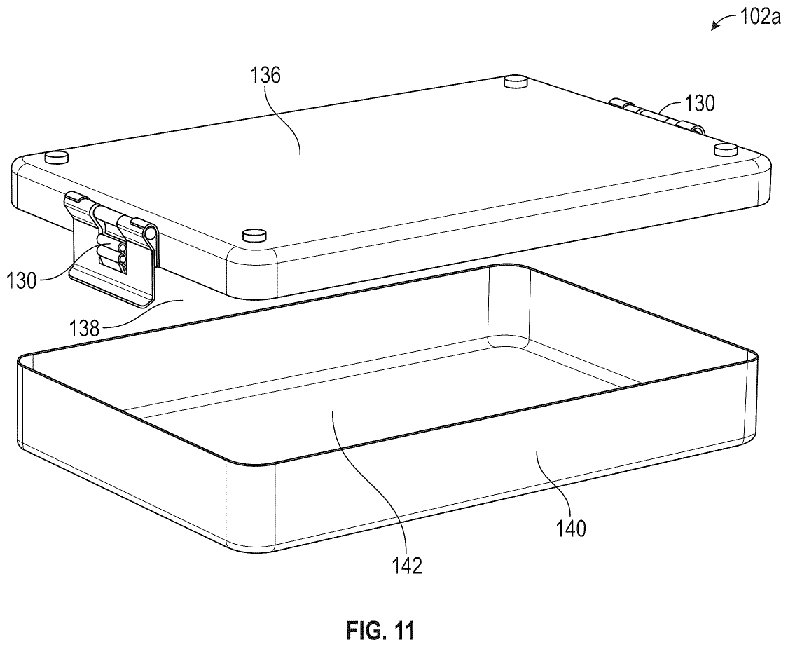

As illustrated in FIG. 10, the first container 102a remains gripped or selectively disposed on the motion axis 116 while the lid 136 is held open. Dental instruments or cassettes 144 may now be loaded into the body 140 via a robotic arm such as the robotic arms 48, 48′, 48″ of the sterilization center 10, a cartesian gantry, a manual or semi-automated mechanism, or a combination thereof.

According to certain embodiments, to close the first container 102a, the motion axis 116 moves upward, pressing the body 140 of the first container 102a back against the stationary lid 136 held by the opening cams 126. Upon full engagement, the first container 102a is closed but not yet latched. In certain embodiments, as the upward motion continues, the latches 130 contact a surface 146 on each of the closing cams 128 as seen in FIG. 9 that are configured to apply a force that rotates the latches 130 closed, securing the lid 136 in place.

According to certain embodiments, after latching, the first container 102a may be fully closed and sealed. The gripper 120 may then be disengaged, for example, a vacuum may be turned off, an electromagnet deactivated, or a mechanical grip may be released. The first container 102a may be retrieved by a robotic arm or gantry, dropped onto a conveyor, or otherwise manually removed, according to an embodiment.

In certain embodiments, the latch opening and closing mechanisms are implemented through fixed cam profiles 132, 146, which apply a mechanical force to the latches 130 during vertical motion of the first container 102a. To facilitate routine maintenance, sanitation, or replacement, the opening and closing cams 126, 128 may be configured to be removable or detachable from their mounting surfaces without requiring disassembly of the entire automated apparatus 100. This ensures compliance with hygiene standards in medical environments and extends the service life of mechanical components.

Similarly, according to certain embodiments, the gripper 120 mechanism, whether it be electromagnetic, vacuum-based, or mechanical, may also be configured for tool-free removal or quick-release disconnection to allow for periodic cleaning, maintenance, or replacement. This modular approach supports sterilization compliance, simplifies upkeep, and minimizes system downtime.

It can be seen therefore that the current automated apparatus 100 provides multiple advantages. For example, the automated apparatus 100 provides full automation where the entire container 102 handling process is automated via a single-axis motion system. The automated apparatus 100 improves safety by eliminating repetitive manual handling, reducing ergonomic risk. Waste reduction may also be provided by replacing disposable wraps and pouches with reusable containers. Additionally, long-term savings may be achieved through reuse of containers and reduced labor, thereby resulting in reduced costs. The automated apparatus 100 may also provide sterility assurance by minimizing contamination risk by automating container handling in a controlled environment.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. An automated system for cleaning or sterilizing at least one instrument, the system comprising:
a plurality of cleaning or sterilization devices configured to clean or sterilize instruments disposed within a work area;
an automated apparatus disposed within the work area that is configured to receive a plurality of containers in a stacked configuration, where the containers are each in turn configured to receive at least one instrument; and
at least one robotic element disposed within the work area;
where the at least one robotic element is configured to:
traverse a length within the work area;
insert or remove the containers to or from the automated apparatus;
transport the at least one instrument between at least two of the plurality of cleaning or sterilization devices; and
operate at least one of the plurality of cleaning or sterilization devices.

2. The system of claim 1, where the at least one robotic element is further configured to insert or remove the at least one instrument from at least one of the containers disposed in the automated apparatus.

3. The system of claim 1, where the automated apparatus comprises:
a frame configured to receive the stacked configuration of the plurality of containers;
a motion axis coupled to the frame and configured to move vertically relative to the frame;
a gripper removably coupled to the motion axis, where the gripper is configured to selectively couple to a first container of the stacked configuration; and
a pair of engagement portions removably disposed on a corresponding pair of lateral ends of the frame,
where the engagement portions are configured to selectively engage with a corresponding pair of latches disposed on the first container.

4. The system of claim 3, further comprising a pair of limiters disposed on either lateral side of the frame and configured to selectively engage with the stacked configuration of containers.

5. The system of claim 4, where each of the limiters comprises:
a linear actuator coupled to the frame; and
a retention rod coupled to the linear actuator.

6. The system of claim 3, where each of the engagement portions comprises:
an opening cam; and
a closing cam disposed above the opening cam.

7. The system of claim 6, where the opening cam comprises a surface configured to engage and apply a rotational force in a first direction to at least one latch of the first container when the first container is moved vertically downward relative to the frame.

8. The system of claim 7, where the closing cam comprises a surface configured to engage and apply a rotational force in a second direction to at least one latch of the first container when the first container is moved vertically upward relative to the frame.

9. The system of claim 3, where the gripper comprises at least one of the following:
a vacuum suction system;
an electromagnet;
a mechanically actuated clamping system; or
a sensor configured to indicate when the first container has been engaged by the gripper.

10. The system of claim 3, where the automated apparatus is connected to an external controller comprising means for operating the automated apparatus when opening or closing the first container.

11. A method for engaging a container for use within an automated system for cleaning or sterilizing medical instruments, the method comprising:
providing a plurality of containers in a stacked configuration;
selectively gripping a first container from the plurality of containers in the stacked configuration;
lowering the first container relative to the remaining plurality of containers in the stacked configuration;
engaging a lid portion of the first container with a pair of engagement portions;
separating the lid portion from a body portion of the first container; and
accessing an interior of the body portion by at least one robotic element of the automated system.

12. The method of claim 11, where selectively gripping the first container from the plurality of containers in the stacked configuration comprises:
contacting a bottom surface of the first container by a gripper removably coupled to a motion axis;
actuating the gripper; and
separating the first container from a remaining plurality of containers by the motion axis,
where the remaining plurality of containers remain in a static position.

13. The method of claim 12, further comprising:
disengaging a pair of limiters before separating the first container from the remaining plurality of containers by the motion axis; and
reengaging the limiters after the motion axis has lowered the first container relative to the remaining plurality of containers by a distance equivalent to at least a height of the first container.

14. The method of claim 11, where engaging the lid portion of the first container with the pair of engagement portions comprises:
receiving a latch disposed on the lid portion by an opening cam of each the engagement portions; and
applying a first rotational force to the latch by the opening cam.

15. The method of claim 14, further comprising:
raising the body portion of the first container to the lid portion;
reengaging the lid portion of the first container with the pair of engagement portions; and
returning the first container to the remaining plurality of containers.

16. The method of claim 15, where reengaging the lid portion of the first container with the pair of engagement portions comprises:
contacting a latch disposed on the lid portion by a closing cam of each of the engagement portions; and
applying a second rotational force to the latch by the closing cam,
where the second rotational force is in an opposing direction relative to the first rotational force.

17. The method of claim 11, where separating the lid portion from a body portion of the first container comprises disposing the lid portion in a static position on the pair of engagement portions while the body portion is moved in a downward direction relative to the lid portion.

18. The method of claim 12, where actuating the gripper comprises at least one of the following:
engaging a vacuum suction system;
actuating an electromagnet; or
actuating a mechanical clamp.

19. The method of claim 11, where providing the plurality of containers in a stacked configuration comprises stacking the plurality of containers in a frame by the at least one robotic element of the automated system.

20. The method of claim 11, where accessing the interior of the body portion by the at least one robotic element of the automated system comprises:
scanning an RFID tag or barcode disposed on at least one cassette or medical instrument by the at least one robotic element; and
inserting or removing the cassette or the at least one medical instrument into or from the interior of the body portion, respectively.
